(19) United States
(12) Patent Application Publication
(10) Pub. No.: US 2020/0330996 A1
(43) Pub. Date: Oct. 22, 2020

(54) SYSTEM AND METHOD FOR OPTIMIZING HEAT TRANSFER FOR TARGET AMPLIFICATION WITHIN A DIAGNOSTIC ASSAY SYSTEM

(71) Applicant: INTEGRATED NANO-TECHNOLOGIES, INC., Henrietta, NY (US)

(72) Inventors: Nathaniel E. Wescott, West Henrietta, NY (US); Richard S. MURANTE, Rochester, NY (US); Dennis M. CONNOLLY, Rochester, NY (US); Mark J. SMITH, Rochester, NY (US)

(73) Assignee: Integrated Nano-Technologies, Inc., Henrietta, NY (US)

(21) Appl. No.: 16/303,441

(22) PCT Filed: Jun. 2, 2017
(86) PCT No.: PCT/US2017/035682

§ 371 Date: Nov. 20, 2018

Related U.S. Application Data
(60) Provisional application No. 62/344,711, filed on Jun. 2, 2016.

(51) Int. Cl. B01L 7/00; C12Q 1/686; G01N 35/00

(52) U.S. Cl. CPC B01L 7/52; G01N 2035/00415; G01N 35/00069; B01L 2200/04; B01L 2300/0681; B01L 2300/0803; B01L 2300/123; B01L 2300/1811; B01L 2300/1816; B01L 2300/1827; B01L 2300/1861; B01L 2400/0622; B01L 2400/0633; G01N 2035/00405; C12Q 1/686

(57) ABSTRACT

A method for optimizing the heat transfer when performing target amplification of an assay fluid, comprising the steps of: (i) moving assay fluid through at least one channel disposed along an underside surface of a disposable cartridge of a diagnostic assay test device such that the fluid collects in a amplification region of the channel; (ii) heating the amplification region of the assay channel to heat the assay fluid; (iii) interposing a conformal material between the underside surface of a disposable cartridge and the RF heater, and (iv) applying a contact pressure between the underside surface of a disposable cartridge and the RF heater.

### CROSS-REFERENCE TO RELATED APPLICATIONS

This application claims priority to U.S. Provisional Patent Application Ser. No. 62/344,711, filed Jun. 2, 2016 entitled “Multi-chamber Rotating Valve and Thermal Control In A Microfluidic Chamber”. The contents of the aforementioned applications are hereby incorporated by reference in their entirety.

This application also relates to International Patent Application No. PCT/US2017/032904, internationally filed May 16, 2017 entitled “Flow Control System for Diagnostic Assay System”, which claims priority to U.S. Provisional Patent Application Ser. No. 62/337,446 filed May 17, 2016 entitled “Multi-Chamber Rotating Valve and Cartridge.” Additionally, this application also relates to U.S. patent application Ser. No. 15/157,584 filed May 18, 2016 entitled “Method and System for Sample Preparation”, which is a continuation of U.S. Non-Provisional patent application Ser. No. 14/056,543, filed Oct. 17, 2013, now U.S. Pat. No. 9,347,086, which claims priority to U.S. Provisional Patent Application Ser. No. 61/715,003, filed Oct. 17, 2012, which is a continuation-in-part of U.S. patent application Ser. No. 12/785,856, filed May 24, 2010, now U.S. Pat. No. 8,663,918, which claims priority to U.S. Provisional Patent Application Ser. No. 61/180,494, filed May 22, 2009, and which is also a continuation-in-part of U.S. patent application Ser. No. 12/754,205, filed Apr. 5, 2010, now U.S. Pat. No. 8,716,006, which claims priority to U.S. Provisional Patent Application Ser. No. 61/158,519, filed Apr. 3, 2009. The contents of the aforementioned applications are hereby incorporated by reference in their entirety.

### FIELD OF THE INVENTION

The present invention relates to a disposable cartridge for a portable diagnostic assay device, and more particularly, to a system and method for heat optimization of reactions in a portable diagnostic assay system.

### BACKGROUND OF THE INVENTION

Fluid analysis of biological samples such as blood and food samples for assay testing general require a series of process steps. These steps generally require that particular fluids contact a reaction area at different times and in varying secession. Furthermore, each fluid may require different pre-treatment prior to contacting the reaction area such as chemical, optical, thermal, mechanical, magnetic or acoustical pre-treatment. A single fluid sample may be subjected to a variety of steps prior to contact with a reaction area such as heating or ultrasonic processing. As the number of fluids and pre-treatment steps increase, the fluid delivery system becomes more complex.

One of the more recent developments in the field of diagnostic testing relates to a portable diagnostic assay device capable of performing a variety of common and complex laboratory procedures without the requirement for a staff of highly-skilled technicians to perform these procedures in a costly laboratory environment/setting. The portable diagnostic assay device and related diagnostic cartridges are disclosed in a portfolio of issued and pending US and foreign patents/patent applications assigned to Integrated Nano-Technologies located in the town of Henrietta, state of New York, USA. The portable diagnostic assay device comprises a small base unit, i.e., generally smaller than a standard briefcase, for accepting one of many distinct, dedicated, and disposable cartridges prepared for conducting a single assay test. For example, the disposable cartridges may be prepared for testing blood borne diseases, food borne bacteria, and/or animal/insect carrying bacteria and viruses.

The diagnostic cartridges comprise a plurality of chambers each containing a reagent used in the assay test, e.g., PCR primers, enzymes and certain chemical compounds. One method to significantly improve the efficiency and yield of PCR amplification is to heat the reaction at various stages in the assay fluid process. The more rapidly an assay fluid reaches a desired temperature, the more efficient is the process. Furthermore, as the accuracy of the temperature improves, the assay sample yield increases which can reduce the number of cycles required to reach a desired level of PCR amplification.

Of the many variables which can impact temperature, the most dominant is the insulating effects of air trapped between assay fluids and the heat source of the diagnostic assay system. This problem is typically resolved by the use of a conductive cream or gel disposed between the heat source and the assay fluid. While this solution may be satisfactory for machines which typically employ greased bearings and gears, it is not well-suited for a portable laboratory requiring the equivalent of a clean-room environment. Furthermore, the portable diagnostic system employs sensitive electronic circuit boards which can be short-circuited should the conductive cream or gel flow into or across the soldered leads of such PC boards. Finally, the conductive cream or gel must be cleaned with each use and, as such, is not practical for most high cycle machines.

A need, therefore, exists for an efficient, reliable, and practical system and method for optimizing the heat transfer associated with target amplification in a portable diagnostic assay system.

### SUMMARY OF THE INVENTION

In one embodiment, a method is provided for optimizing the heat transfer when performing target amplification of an assay fluid, comprising the steps of: (i) moving assay fluid through at least one channel disposed along an underside surface of a disposable cartridge of a diagnostic assay test device such that the fluid collects in an amplification region of the channel; (ii) heating the amplification region of the assay channel to heat the assay fluid; (iii) interposing a conformal material between the underside surface of a disposable cartridge and the RF heater, and (iv) applying a contact pressure between underside surface of a disposable cartridge and the RF heater.

In another embodiment, a diagnostic assay system is provided including a mounting platform receiving a disposable cartridge, a heat source disposed in combination with the mounting platform and a multi-axis actuation system operative to rotationally index the cartridge rotor relative to the cartridge body and apply a contact force along a mating interface disposed between the underside surface of the cartridge and the heat source.

### BRIEF DESCRIPTION OF THE DRAWINGS

The present invention is disclosed with reference to the accompanying drawings, wherein:

FIG. 1 is a perspective view of a portable diagnostic assay system operative to accept one of a plurality of disposable cartridges configured to test samples of collected blood/food/biological materials.

FIG. 2 is an exploded perspective view of one of the disposable cartridges configured to test the blood/food/biological materials.

FIG. 3 is a top view of the one of the disposable cartridges illustrating a variety of assay chambers including a central assay chamber for receiving the blood/food/biological material and at least one other chamber containing an assay chemical suitable to breakdown the blood/food/biological material to detect a particular attribute thereof.

FIG. 4 is a bottom view of the disposable cartridge shown in FIG. 3 illustrating a variety of channels operative to move at least a portion of the assay material from one chamber to another for the purpose of performing multiple operations on the sample.

FIG. 5 is a perspective view of a portable diagnostic assay system and an exploded view of the requisite components necessary for optimizing target amplification including a mounting platform having a mounting plate, a heat source integrated within the mounting plate, a conductive conformal layer disposed over the mounting plate and a multi-axis actuation system operative to apply a threshold contact force/pressure at a mating interface between the conductive conformal and a fluid channel disposed on an underside surface of the disposable cartridge.

FIG. 6 depicts a profile view of the portable diagnostic assay system depicted in FIG. 5 including a schematic view of the cartridge rotor, the mounting platform, heat source, conformal conductive sheet and the multi-axis actuation system.

FIGS. 7 and 8 depict a schematic view of the multi-axis actuation system of the portable diagnostic assay system moving between an open or disengaged position (FIG. 7) and a closed or engaged position (FIG. 8).

FIG. 9 depicts an enlarged view of the actuation plate together with the conformal conductive elastomeric material disposed over the actuation plate.

FIG. 10 is an enlarged bottom view of the disposable cartridge showing the underside surface thereof including a pair of assay channels for target amplification together with a film of polyurethane material disposed over the assay channels.

FIG. 11 depicts an enlarged cross-sectional view taken substantially along lines 11-11 of FIG. 7.

FIG. 12 depicts an enlarged cross-sectional view taken substantially along lines 12-12 of FIG. 8.

Corresponding reference characters indicate corresponding parts throughout the several views. The examples set out herein illustrate several embodiments of the invention but should not be construed as limiting the scope of the invention in any manner.

### DETAILED DESCRIPTION

A disposable cartridge is described for use in a portable/automated assay system such as that described in commonly-owned, co-pending U.S. patent application Ser. No. 15/157,584 filed May 18, 2016 entitled “Method and System for Sample Preparation” which is hereby included by reference in its entirety. While the principal utility for the disposable cartridge includes DNA testing, the disposable cartridge may be used to detect any of a variety of diseases which may be found in either a blood, food or biological specimen. For example, blood diagnostic cartridges may be dedicated cartridges useful for detecting hepatitis, autoimmune deficiency syndrome (AIDS/HIV), diabetes, leukemia, graves, lupus, multiple myeloma, etc., just naming a small fraction of the various blood borne diseases that the portable/automated assay system may be configured to detect. Food diagnostic cartridges may be used to detect salmonella, E-coli, Staphylococcus aureus or dysentery. Blood diagnostic cartridges may be dedicated cartridges useful for detecting insect or animal borne diseases including malaria, encephalitis and the West Nile virus.

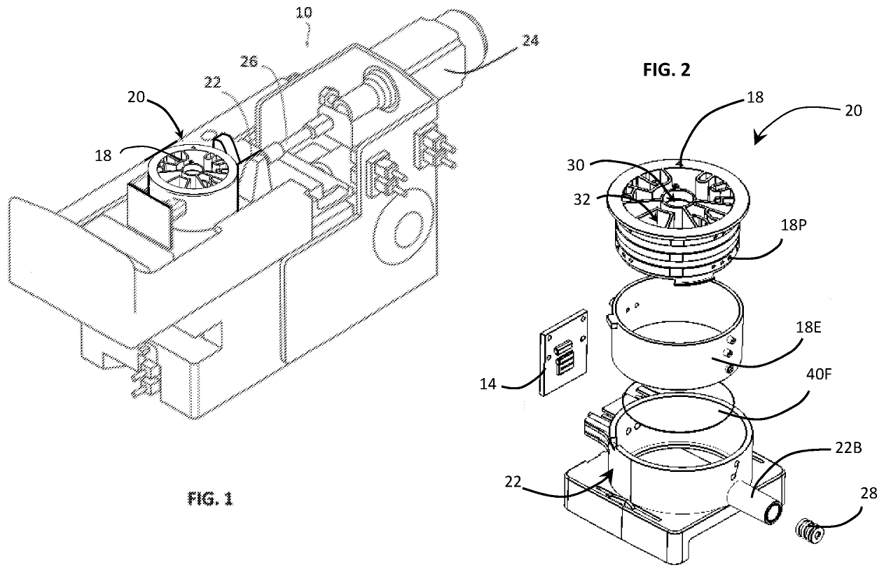

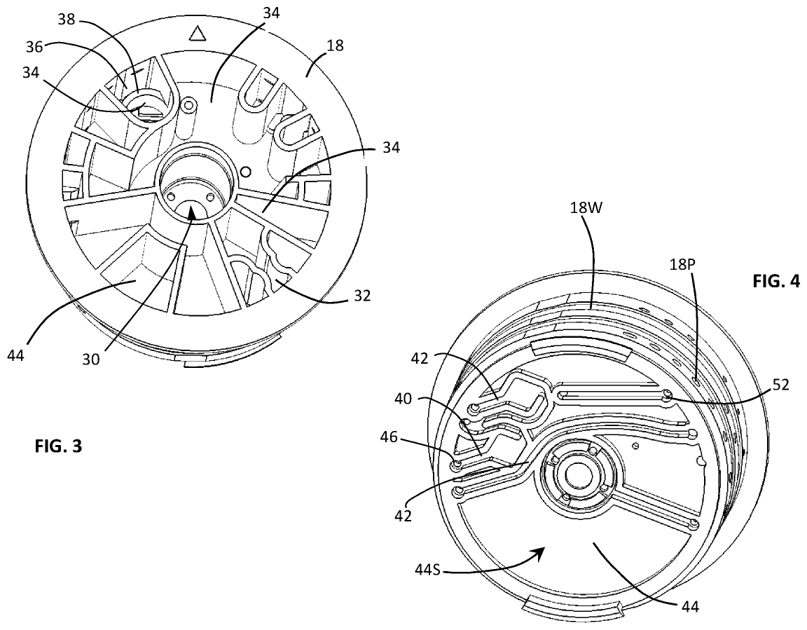

More specifically, and referring to FIGS. 1 and 2, a portable assay system 10 receives any one of a variety of disposable assay cartridges 20, each selectively configured for detecting a particular attribute of a fluid sample, each attribute potentially providing a marker for a blood, food or biological (animal borne) disease. The portable assay system 10 includes one or more linear and rotary actuators operative to move fluids into, and out of, various compartments or chambers of the disposable assay cartridge 20 for the purpose of identifying or detecting a fluid attribute. More specifically, a signal processor 14, i.e., a PC board, controls a rotary actuator (not shown) of the portable assay system 10 so as to align one of a variety of ports 18P, disposed about a cylindrical rotor 18, with a syringe barrel 22B of a stationary cartridge body 22. The processor 14 controls a linear actuator 24, to displace a plunger shaft (not shown) so as to develop pressure, i.e., positive or negative (vacuum) in the syringe barrel 22. That is, the plunger shaft displaces an elastomer plunger 28 within the syringe 22 to move and/or admix fluids contained in one or more of the chambers 30, 32.

The disposable cartridge 20 provides an automated process for preparing the fluid sample for analysis and/or performing the fluid sample analysis. The sample preparation process allows for disruption of cells, sizing of DNA and RNA, and concentration/clean-up of the material for analysis. More specifically, the sample preparation process of the instant disclosure prepares fragments of DNA and RNA in a size range of between about 100 and 10,000 base pairs. The chambers can be used to deliver the reagents necessary for end-repair and kinase treatment. Enzymes may be stored dry and rehydrated in the disposable cartridge, or added to the disposable cartridge, just prior to use. The implementation of a rotary actuator allows for a single plunger to draw and dispense fluid samples without the need for a complex system of valves to open and close at various times. This greatly reduces potential for leaks and failure of the device compared to conventional systems. It will also be appreciated that the system greatly diminishes the potential for human error.

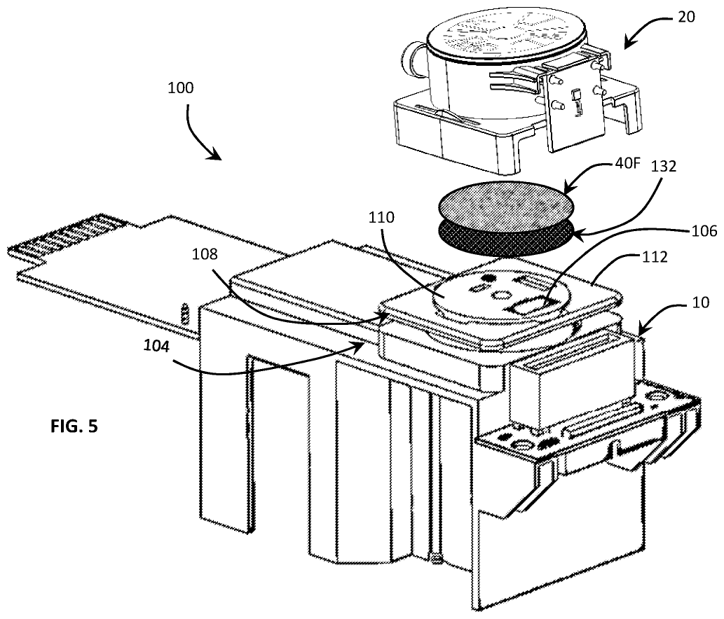

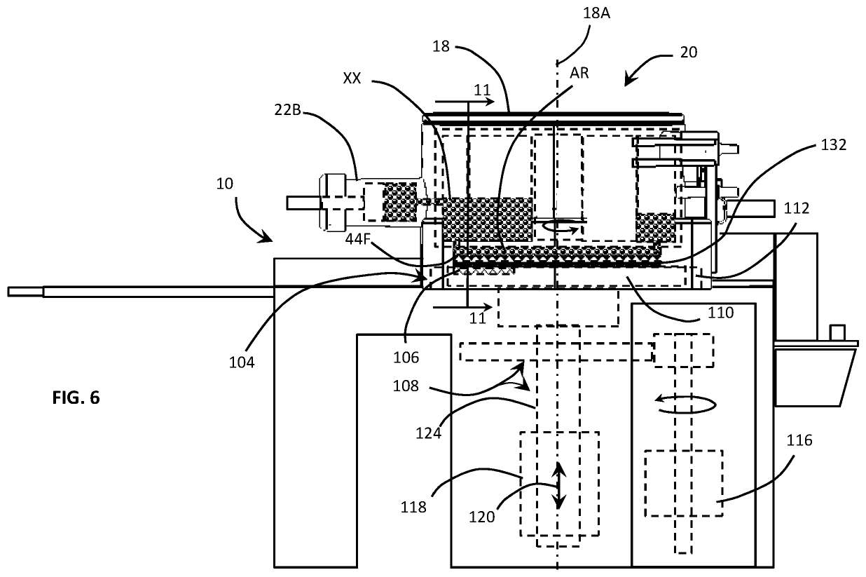

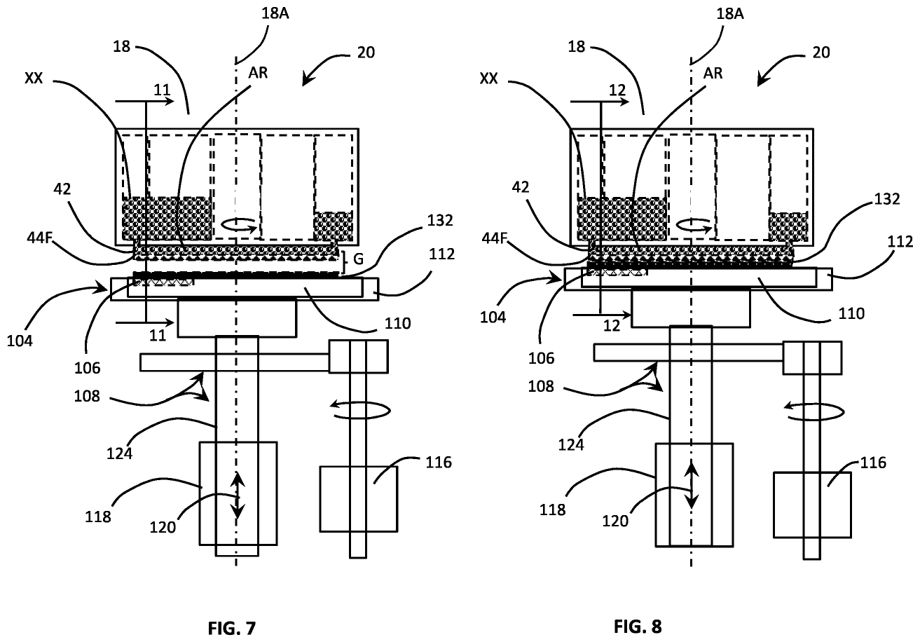

In FIGS. 3 and 4, the cylindrical rotor 18 includes a central chamber 30 and a plurality of assay chambers 32, 34 surrounded, and separated by, one or more radial or circumferential walls. In the described embodiment, the central chamber 30 receives the fluid sample while the surrounding chambers 32, 34 may contain a premeasured assay chemical or reagent for the purpose of detecting an attribute of the fluid sample. The chemical or reagents may be initially dry and rehydrated immediately prior to conducting a test. Some of the chambers 32, 34 may be open to allow the introduction of an assay chemical while an assay procedure is underway or in process. The chambers 30, 32, 34 are disposed in fluid communication, e.g., from one of the ports 18P to one of the chambers 30, 32, 34, by channels 40, 42 molded along a bottom panel 44, i.e., along underside surface of the rotor 18.

Depending upon the specific function of the cartridge 20, one important feature of the channels 40, 42 is to facilitate and augment amplification by forming a region which may be heated from the underside of the cartridge 20. During development of the disposable cartridge and diagnostic assay system, the inventors were faced with various challenges associated with accelerating amplification. More specifically, the inventors learned that the use of conventional conductive grease along the mating interface of a channel 42 was inadequate to reach a desired temperature set point, i.e., to transfer heat, within a reasonable time frame. It was at this point that the inventors began conducting a variety of inventive methods and configurations which would lead to a two-fold increase in amplification time. These tests/inventive discoveries are discussed in the subsequent paragraphs.

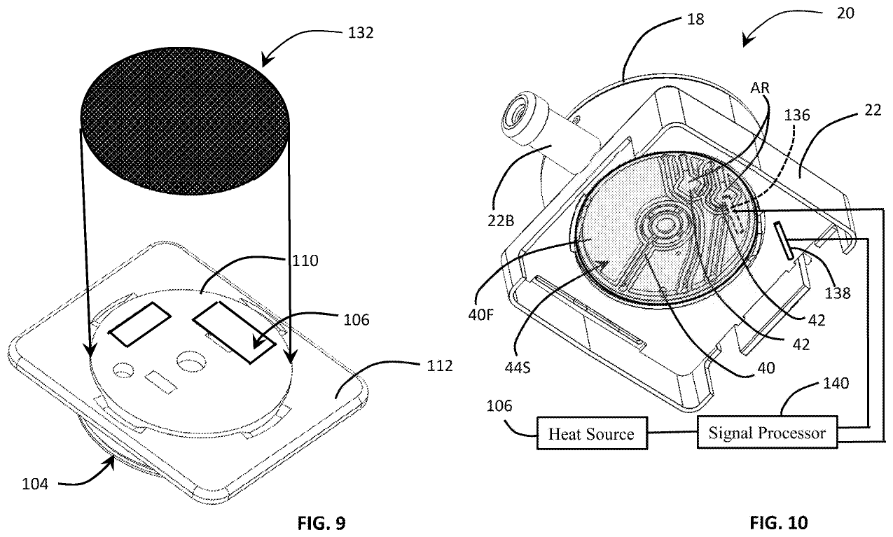

In FIGS. 5, 6, 9 and 10 a diagnostic assay system 100 comprises: (i) a mounting platform 104 configured to receive a disposable cartridge 20; (ii) a heat source 106 integrated within mounting platform 104, and (iii) an actuation system 108 configured to move a plate 112 of the mounting platform 104 into contact with an underside surface of the disposable cartridge. With respect to the latter, the actuation system 108 may rotationally index the rotor 18 of the cartridge 20 into alignment with the syringe barrel of the cartridge body while also displacing the plate 112 into contact with the underside surface of the cartridge 20. More specifically, the mounting platform 104 includes a circular disc 110 disposed at the center of a rectangular or square mounting plate 112. The circular disc 110 is adjacent to and is contiguous with the underside surface 44S (best seen in FIG. 10) of the disposable cartridge 20. As mentioned in the preceding paragraph, the underside surface 44S of the disposable cartridge 20 forms a network of channels 40, 42, at least one of which facilitates target amplification by providing a region AR (FIG. 10) which enhances heat transfer. Specifically, at least one of the channels 42 opens-up or diverges into an accumulation region AR pocket where amplification can occur by rapidly heating the region to a desired or threshold temperature. This amplification region AR is covered by a film 44F of plastic, however, any suitably thin, low resistivity material will suffice to provide a mating interface for heat transfer, i.e., between the amplification region AR and the circular disc 110.

In the described embodiment, the heat source 106 is integrated within the circular disc 106 of the mounting plate 104. The heat source 106 may be any resistive heater, however, in the disclosed embodiment, a low wattage RF heat source or inductive heater may be employed. That is, inasmuch as the diagnostic assay tester 10 is portable, a source of high current may not be readily available. In view of these contingencies, an RF and/or inductive heater may be preferable inasmuch as such heat sources may operate on 6-12 volt battery power. A typical RF heating device may include any strip of material which is responsive to RF energy. Such materials include a molecular lattice which is excited, i.e., vibrates, in the presence of an RF energy field within a particular frequency band.

In FIGS. 6, 7 and 8, the multi-axis actuation system 108 integrates with the mounting platform 104 and comprises: (i) a rotary actuator 116 for rotationally indexing the cartridge rotor 18 of the disposable cartridge 20, and a linear actuator 118 operative to apply a contact force/pressure parallel to the rotational axis 18A of the cartridge rotor. While the rotary actuator 116 is shown driving the rotor 18 by pinion/spur gear combination along an axis parallel to the rotational axis 18A of the rotor 18, it will be appreciated that other drive systems are contemplated. For example, greater accuracy and control may be provided by a worm gear (not shown) having an axis perpendicular to the rotational axis 18A. The linear actuator 118 drives a shaft 124 along the rotational axis 18A to induce a contact pressure along a mating interface between the underside surface 44S of the cartridge rotor 18 and the mounting plate 112. FIG. 7 shows the multi-axis actuation system 108 in an open or unengaged position such that the underside surface 44S of the cartridge rotor 18, or the amplification channel 42, is separated from the mounting plate 112 by a gap G. FIG. 8 depicts the multi-axis actuation system 108 in a closed or engaged position such that the mounting plate 112 moves upwardly toward the underside surface 44S of the cartridge rotor 18 until the mounting plate 112, along with the integrated heater 106, is pressed against the fluid channel 42.

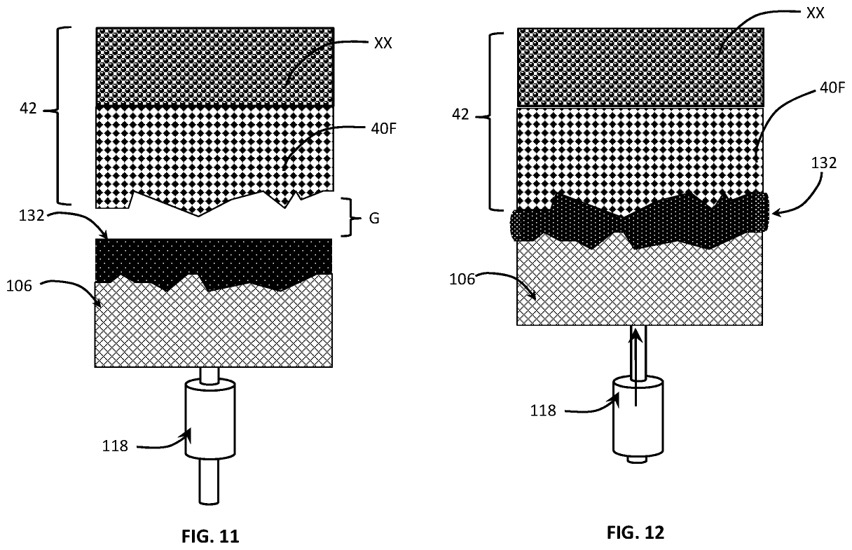

In FIGS. 5, 6, and 9-12, the inventors discovered that a number of factors dramatically altered the efficiency and time for target amplification of the sample fluid. In one embodiment, the inventors discovered that by imposing a small contact pressure along the mating interface between the amplification channel 42 and the heat source 106, the cycle time required for target amplification was significantly reduced. Additionally, and in another embodiment, it was determined that the addition of a thin layer of conformal material 130, i.e., on the order of between two (2) to four (4) mils., also filled pockets of air caused by surface roughness along the mating interface. FIG. 11 depicts an enlarged view of the heat source 106, the channel 42 (comprising the thin film layer 40F which covers the fluid XX), and the conformal layer 132 disposed therebetween. FIG. 12 depicts the same components as those depicted in FIG. 11, but for the linear actuator 118 closing the gap G and imposing a threshold contact pressure along the mating interface. There, it will be appreciated that the small pockets of air generated by the irregular surface of the mating interface, are filled by the conformal layer. As such, heat flows unabated by the insulating pockets of air. In the described embodiment, the threshold contact pressure may be within a range of between about 0.25 lbs./in.² to about 7 lbs./in². More preferably, the threshold contact pressure may be within a range of between about 0.25 lbs./in.² to about 3 lbs./in². Conformal materials which may be used include silicones, elastomers, rubbers, urethanes and films having a low Young's modulus, a high percent elongation (i.e., high strain properties) or a low durometer. With respect to the latter, materials having a Shore-A hardness of less than about 75 may be useful for practicing the inventive features of the disclosure.

Testing of the various configurations described herein provides nearly a two-fold increase in temperature response and accuracy. For most of the assay fluid procedures, temperatures can be controlled to within one degree Celsius (1°). In one embodiment, a thermocouple 136 may be introduced to measure the temperature within the amplification region AR while another thermocouple 138 reads an ambient temperature to establish a baseline or threshold temperature. The thermocouple 136 in the amplification region AR issues an actual temperature signal indicative of an instantaneous temperature of the assay fluid XX. The signal processor 140 is responsive to the actual temperature signal, compares it to a stored threshold temperature signal, and controls the heat source such that the actual temperature is maintained within a threshold range of the threshold temperature. Alternatively, a second thermocouple 138 issues a baseline or ambient temperature signal for comparison to the actual temperature signal. While the illustrated embodiment depicts a thermocouple along the underside surface of the disposable cartridge 20, it will be appreciated that one or both of the thermocouples 136, 138 may be disposed in combination with the contact plate 112, proximal the heat source 106 and juxtaposed the underside of the cartridge rotor 18.

While the invention has been described with reference to particular embodiments, it will be understood by those skilled in the art that various changes may be made and equivalents may be substituted for elements thereof without departing from the scope of the invention. In addition, many modifications may be made to adapt a particular situation or material to the teachings of the invention without departing from the scope of the invention.

Therefore, it is intended that the invention not be limited to the particular embodiments disclosed as the best mode contemplated for carrying out this invention, but that the invention will include all embodiments falling within the scope and spirit of the appended claims.

1. A diagnostic assay system including a disposable cartridge operative to perform target amplification of nucleic acids, comprising:
a rotary drive mechanism operative to index a cartridge rotor about a rotational axis such that a port of the cartridge rotor aligns with a barrel port of a syringe barrel to receive or withdraw a reagent fluid into or out of an assay chamber of the cartridge rotor, the cartridge rotor having at least one channel along the underside surface of the cartridge rotor operative to move assay fluids through the channel from one assay chamber to another assay chamber and having a region for performing target amplification on the assay fluids as the assay fluids pass through the at least one channel;
a plate juxtaposed with the region for performing target amplification on the assay fluids as they move from one chamber to another assay chamber;
a heat source integrated with the plate and operative to transfer heat into the region for performing target amplification on the assay fluids, and
a linear actuator connected to the linear plate and operative to induce a linear force parallel to the rotational axis and perpendicular to an underside surface of the cartridge rotor.

2. (canceled)

3. The diagnostic assay system of claim 1 further comprising a conformal material disposed between an underside surface of the cartridge rotor and the plate.

4. The diagnostic assay system of claim 3 wherein the conformal material is loaded with a conductive material.

5. The diagnostic assay system of claim 1 wherein the heat source comprises an Radio Frequency (RF) energy source for emitting a field of RF energy and at least one strip of material disposed on a face of the plate which is heated in response to exposure to the RF energy field.

6. The diagnostic assay system of claim 1 wherein the heat source comprises an inductive energy source for emitting a field of electromagnetic energy and at least one strip of material disposed on a face of the plate which is heated in response to exposure to the field of inductive energy.

7. The diagnostic assay system of claim 1 further comprising a signal processor and a thermocouple disposed internally of the region for performing target amplification on the assay fluids, the thermocouple issuing an actual temperature signal indicative of an instantaneous temperature of the and wherein the signal processor compares the actual temperature signal to a threshold temperature signal to control the heat source such that the actual temperature is maintained within a threshold range of the threshold temperature.

8. The diagnostic assay chamber of claim 3 wherein the conformal material has a Shore-A hardness less than about 75.0.

9. A method for optimizing the heat transfer when performing target amplification of an assay fluid, comprising the steps of:
moving assay fluid through at least one assay channel disposed along an underside surface of a disposable cartridge of a diagnostic assay test device such that the assay fluid collects in a amplification region of the assay channel;
heating the amplification region of the assay channel by a heater to elevate the temperature of the assay fluid;
interposing a conformal material between the assay channel and the heater; and
applying a contact pressure between the heater and the assay channel.

10. The method of claim 9 wherein the step of heating the amplification region includes the step of heating a strip disposed on a mounting plate proximal to the amplification region of an assay channel.

11. The method of claim 9 wherein the step of heating the amplification region includes the step of inductive heating a strip disposed on a mounting plate proximal to the amplification region of an assay channel.

12. The method of claim 9 wherein the step of interposing a conformal material further comprises the step of suspending a conductive material in the conformal material.

13. The method of claim 9 wherein the step of heating comprises the step of: integrating a strip of material into the plate which is responsive to RF energy to heat the strip.

14. The method of claim 9 wherein the contact pressure is within a range of between about 0.25 lbs./in.² to about 7 lbs./in².

15. The method of claim 9 wherein the conformal material includes materials from the group of: silicones, urethanes, rubbers, elastomers, and films.

16. A diagnostic assay system, comprising:
a mounting platform receiving a disposable cartridge comprising a cartridge body rotationally mounting a cartridge rotor about an axis, the cartridge rotor having at least one channel disposed along the underside surface of the cartridge rotor for moving fluids into and out of at least one assay chamber, the channel and platform defining a mating interface facilitating heat exchange to and from the assay fluids within the channel while conducting a target amplification procedure,
a heat source disposed in combination with the mounting platform, disposed adjacent to the heat exchange surface and across the mating interface thereof, and
an actuation system operative to rotationally index the cartridge rotor relative to the cartridge body and apply a contact force along the rotational axis of the cartridge rotor to induce a contact pressure across the mating interface.

17. The diagnostic assay system of claim 16 further comprising: a conformal material between the assay channel and the heat source to improve heat transfer.

18. The diagnostic assay system of claim 16 wherein the conformal material further comprises a conductive material suspended within the conformal material.

19. The diagnostic assay system of claim 16 further comprising a strip of material disposed in the mounting platform along the mating interface.

20. The diagnostic assay system of claim 16 wherein the contact pressure is within a range of between about 0.25 lbs./in.² to about 7 lbs./in².

21. The diagnostic assay system of claim 16 wherein the heat source is a Radio Frequency (RF) heat source.
